(19) United States
(12) Patent Application Publication
(10) Pub. No.: US 2026/0357299 A1
(43) Pub. Date: Nov. 12, 2026

(54) MICROBACTERIUM STRAIN AND USE THEREOF IN LITHIUM ENRICHMENT AND SEPARATION FROM LOW-GRADE BRINE

(71) Applicant: SHANDONG UNIVERSITY, Jinan (CN)

(72) Inventors: Weizhi ZHOU, Jinan (CN); Fei HAN, Jinan (CN); Jianhua LEI, Jinan (CN)

(21) Appl. No.: 19/560,097

(22) Filed: Mar. 8, 2026

(30) Foreign Application Priority Data
Mar. 11, 2025 (CN) 202510284911X

(51) Int. Cl. C12P 3/00; C12N 1/20; C12R 1/265

(52) U.S. Cl. CPC C12P 3/00; C12N 1/20; C12R 2001/265

(57) ABSTRACT

The present disclosure belongs to the technical field of microorganisms and environmental resource utilization, and relates to a method for enriching and separating lithium from lithium-containing aqueous medium using a microbial agent. The microbial agent comprises viable cells of Microbacterium paraoxydans HB04. The strain was deposited on Feb. 17, 2025 at the China Center for Type Culture Collection (CCTCC) under accession number CCTCC NO: M 2025230. The microbial agent can achieve adsorption and accumulation of lithium within relatively broad ranges of temperature, pH and salinity, and is applicable to lithium enrichment and separation from a lithium-containing aqueous medium, such as low-grade brine systems (including oilfield produced water and heavy-oil produced water).

### CROSS-REFERENCE TO RELATED APPLICATIONS

The present disclosure claims priority to Chinese Patent Application No. CN202510284911.X, filed on Mar. 11, 2025, entitled “Microbacterium Strain and Use Thereof in Lithium Enrichment and Separation from Low-Grade Brine”. The entire contents of the above application are incorporated herein by reference for all purposes.

### TECHNICAL FIELD

The present disclosure belongs to the technical field of microorganisms and environmental protection, and in particular relates to a Microbacterium strain and its application in lithium enrichment and separation from a lithium-containing aqueous medium (e.g., low-grade brine, oilfield produced water, and lithium-containing culture media/broths).

### BACKGROUND OF THE INVENTION

The information described in this Background section is provided only to facilitate understanding of the technical background of the present disclosure, and should not be construed as an admission of the prior art or common general knowledge.

Lithium is a metal widely regarded as having high electrochemical activity, and is a strategic metal of great significance in the 21st century as well as a promising new-energy metal. The rise of the new-energy industry has significantly increased the demand for lithium batteries, and the imbalance between supply and demand of lithium resources has led to a rapid increase in the price of lithium. In addition to providing power for electric vehicles, lithium-ion batteries are also critical for grid energy storage associated with renewable energy sources such as wind and solar, thereby accelerating carbon neutrality and reducing carbon emissions. Forecasts indicate that from 2023 to 2050, lithium demand will increase by 8 to 10 times; therefore, extracting lithium resources through unconventional pathways have also attracted attention. Low-quality brines are important potential lithium resources, with abundant reserves and wide geographic distribution. Among them, oilfield produced water is a typical representative of low-quality brine. Recovering lithium from oilfield produced water has potential, which may help solve the imbalance between supply and demand of lithium resources.

The enrichment and recovery of useful metals from low-grade brine by special microorganisms has long been a hot topic. Compared with aqueous lithium separation methods such as solvent extraction and ion exchange, which are energy-intensive and produce by-products, bacterial extraction of lithium is considered a feasible, non-toxic, low-cost method and an effective alternative. One notable advantage of microbial processes is their ability to operate under mild conditions, which effectively simplifies downstream processing. In some cases, special bacteria can produce abundant extracellular polymers through their metabolic activity to selectively adsorb and enrich lithium from various other ions in produced water. These special microorganisms are usually extremophiles that exist and grow in environments with extreme temperatures, extreme pH conditions, or high salinity; such remarkable adaptability enables them to function in oil produced water as well. However, there remains further development of available strains for lithium enrichment and separation in low-grade brine systems (e.g., heavy-oil produced water).

### SUMMARY OF THE INVENTION

The present disclosure aims to provide a Microbacterium strain capable of growing within relatively broad ranges of temperature, pH, and salinity, and capable of specifically and selectively adsorbing and/or accumulating lithium in lithium-containing aqueous media. The present disclosure further provides applications of the strain for lithium enrichment and separation from lithium-containing aqueous media, including low-grade brine systems such as oilfield produced water (e.g., heavy-oil produced water) and other complex brine systems.

To achieve the above objective, the present disclosure adopts the following technical solutions:

In one aspect, the present disclosure provides a Microbacterium strain, named Microbacterium paraoxydans HB04. The strain was deposited on Feb. 17, 2025 at the China Center for Type Culture Collection (CCTCC, address: 299 Bayi Road, Wuchang District, Wuhan City, Hubei Province, China), under accession number CCTCC NO: M 2025230.

In another aspect, the present disclosure provides a microbial agent/microbial preparation, which comprises the Microbacterium strain described in the above first aspect, or a fermentate thereof, or metabolic products thereof. In some embodiments, the metabolic products comprise extracellular polymers.

In yet another aspect, the present disclosure provides use of the above Microbacterium strain or the above microbial agent as a lithium enrichment agent, and use thereof for selective lithium enrichment and separation in a lithium-containing environment.

Since the Microbacterium in the present disclosure can grow and reproduce at 10-50 °C, pH 4.0-11.0, and salinity 0-7%, and can specifically enrich lithium to a certain extent, it can adapt to various harsh lithium-containing environments, such as high-temperature or low-temperature environments, acidic or alkaline environments, and low-salinity or high-salinity environments.

In still another aspect, the present disclosure provides a method for enriching and separating lithium from a lithium-containing aqueous medium. The method comprises: contact-culturing a lithium-containing aqueous medium with a microbial agent comprising viable cells of strain HB04, under conditions suitable for growth or metabolism of the strain, such that lithium is adsorbed onto the cells, accumulated within the cells, or both; and after cultivation, separating the cells from the liquid phase to obtain cells onto which lithium has been adsorbed, cells within which lithium has been accumulated, or both. In some embodiments, the lithium-containing aqueous medium is a brine, such as oilfield produced water, and may in particular be heavy-oil produced water. In other embodiments, the lithium-containing aqueous medium is a lithium-containing culture medium or culture broth (e.g., LB medium supplemented with a soluble lithium salt to provide detectable lithium ions (Li⁺)).

Beneficial effects of the above technical solutions:

Microbacterium paraoxydans HB04 can grow and reproduce within the ranges of 10-50 °C, pH 4.0-11.0, and salinity 0-7%, and can enrich lithium to a certain extent; moreover, under conditions of 10-40 °C, pH 5.0-8.0, and salinity 0-5%, the lithium accumulation amount and enrichment rate show almost no significant attenuation, thereby providing a feasible microbial technical route for lithium recovery in complex brine systems.

### BRIEF DESCRIPTION OF DRAWINGS

The drawings in the specification, which constitute a part of the present disclosure, are used to provide further understanding of the present disclosure. The schematic embodiments of the present disclosure and the descriptions thereof are used to explain the present disclosure, and do not constitute any improper limitation of the present disclosure.

FIG. 1A is a photograph of colonies of strain HB04 cultured on a plate.

FIG. 1B is an enlarged partial view of FIG. 1A.

FIG. 2 is an optical microscope image of strain HB04.

FIG. 3 shows a phylogenetic tree of strain HB04.

FIG. 4A shows a growth curve of strain HB04 at different initial Li⁺ concentrations.

FIG. 4B shows lithium enrichment capability of strain HB04 at different initial Li⁺ concentrations.

FIG. 5A shows a growth curve of strain HB04 at different salinities.

FIG. 5B shows lithium enrichment capability of strain HB04 at different salinities.

FIG. 6A shows a growth curve of strain HB04 at different temperatures.

FIG. 6B shows lithium enrichment capability of strain HB04 at different temperatures.

FIG. 7A shows a growth curve of strain HB04 at different pH values.

FIG. 7B shows lithium enrichment capability of strain HB04 at different pH values.

FIG. 8A shows growth curves of strain HB04 in heavy-oil produced water sample 1 and sample 2.

FIG. 8B shows enrichment capability of strain HB04 in heavy-oil produced water sample 1 and sample 2.

### DESCRIPTION OF THE INVENTION

It should be noted that the following detailed description is exemplary and is intended to provide further illustration of the present disclosure. Unless otherwise specified, the technical and scientific terms used herein have the same meanings as commonly understood by those of ordinary skill in the art to which the present disclosure belongs.

When the term “comprise” and/or “include” is used in the present disclosure, it indicates the presence of a feature, step, operation, device, component and/or combination thereof, but does not exclude the presence or addition of other features, steps, operations, devices, components and/or combinations thereof.

### Definitions of Terms

(1) Cells:

As used in the present disclosure, “cells” generally refer to microbial cellular material. The cells obtained after contact cultivation may include viable cells that remain active and inactive cells formed during cultivation (e.g., deactivated, dead, or partially damaged cells).
(2) Viable cells:

As used in the present disclosure, “viable cells” refer to cells that still have the ability to grow and reproduce under suitable cultivation conditions (e.g., capable of forming colonies).
(3) Fermentate/fermentation broth:

The term “fermentate” refers to fermentation products obtained by fermentation cultivation. The corresponding fermentate may be a liquid system obtained during fermentation cultivation of strain HB04, and thus may also be referred to as fermentation broth or culture broth. The liquid may contain cells, but does not necessarily need to contain cells; the liquid preferably contains metabolic products produced by strain HB04.
(4) Metabolic products:

In the present disclosure, the term “metabolic products” may include extracellular metabolic products and/or intracellular metabolic products produced by strain HB04 during cultivation. In some embodiments, the metabolic products may comprise extracellular polymers.
(5) Supernatant:

In embodiments of the present disclosure, a fermentation broth or culture broth containing cells is subjected to solid-liquid separation by centrifugation, filtration, sedimentation, or other means known in the art. After removal of the cells, the remaining liquid may be referred to as a “supernatant”. In some embodiments, the supernatant may contain extracellular metabolic products of strain HB04.
(6) Cell lysate and cell-free extract:

In embodiments of the present disclosure, disrupting separated cells can yield a cell lysate; the disruption method may be ultrasonication (e.g., ultrasonication in an ice bath to disrupt cells) or other means known in the art. Further, the cell lysate may be centrifuged and a supernatant may be collected, and such supernatant may be referred to as a “cell-free extract”. In some embodiments, the cell lysate or cell-free extract may contain intracellular metabolic products of strain HB04.
(7) Microbial agent for implementing the method of the present disclosure:

In the present disclosure, the term “microbial agent” refers to a microbial preparation used to implement the lithium enrichment and separation method of the present disclosure.

In some embodiments, the microbial agent may comprise the cells of strain HB04and/or a fermentate thereof and/or metabolic products thereof; and may further comprise, as additional component(s), one or more selected from a supernatant, a cell lysate, and a cell-free extract.

It should be understood that the additional components such as the fermentate, supernatant, and cell-free extract, may themselves be liquid systems that contain cells or do not contain cells.

To facilitate implementation of the lithium enrichment and separation method of the present disclosure (including the steps of contact cultivation and, after contact cultivation, solid-liquid separation to obtain lithium-containing cells (i.e., cells onto which lithium has been adsorbed and/or within which lithium has been accumulated)), in some embodiments, the microbial agent used to implement the method comprises viable cells of strain HB04, and may further comprise one or more of the above additional components. In such viable-cell embodiments, the microbial agent as a whole still comprises viable cells.
(8) Lithium-containing aqueous medium:

As used herein, the term “lithium-containing aqueous medium” refers to an aqueous liquid medium containing lithium ions (Li⁺). In some embodiments, the lithium-containing aqueous medium includes, but is not limited to, brines (e.g., oilfield produced water, such as heavy-oil produced water) and lithium-containing culture media or culture broths (e.g., LB medium supplemented with a soluble lithium salt).

### Strain Information and Deposit

Microbacterium paraoxydans HB04 of the present disclosure was isolated from Antarctic sea ice, and is an aerobic Gram-positive bacterium. The colonies are circular, light yellow, opaque, full in morphology, smooth and moist, and are easily picked up; the bacterial lawn grows along streak lines. The cells are rod-shaped; some are arranged at certain angles, and two cells may form a V-shape.

Based on 16S rDNA sequencing and comparison with type strains, in combination with phylogenetic tree analysis, the strain was determined to be Microbacterium paraoxydans of the genus Microbacterium, and was named Microbacterium paraoxydans HB04.

The strain was deposited on Feb. 17, 2025 at the China Center for Type Culture Collection (CCTCC, address: 299 Bayi Road, Wuchang District, Wuhan City, Hubei Province, China), under accession number CCTCC NO: M 2025230.

For convenience of description, Microbacterium paraoxydans HB04 may also be abbreviated as strain HB04 or HB04 in this specification.

### Preparation of Microbial Agent

The present disclosure further provides a microbial agent, the active component(s) of which may be strain HB04 itself, a fermentation broth/culture broth containing cells, a supernatant obtained after removal of cells, a cell lysate, a cell-free extract, or metabolic products produced by strain HB04, and the like. The metabolic products may comprise extracellular polymers.

In some embodiments, the metabolic products comprise extracellular polymers of the strain. Microbacterium HB04 can produce abundant extracellular polymers through its metabolic activity to exhibit, to a certain extent, preferential adsorption/enrichment of lithium from complex ionic systems such as produced water.

Further, in embodiments of the present disclosure, the microbial agent may be prepared as follows: inoculating strain HB04 into LB medium and culturing, thereby obtaining the microbial agent.

In some embodiments, in addition to the active component(s), the microbial agent may further comprise a carrier. The carrier may be a carrier commonly used in the microbial agent field and biologically inert. The carrier may be a solid carrier or a liquid carrier.

The solid carrier may be a mineral material, a plant material, or a polymer compound. The mineral material may be at least one selected from clay, talc, kaolin, montmorillonite, white carbon (precipitated silica), zeolite, quartz, and diatomite. The plant material may be at least one selected from corn flour, soybean flour, and starch. The polymer compound may be polyvinyl alcohol and/or polyethylene glycol.

The liquid carrier may be an organic solvent, vegetable oil, mineral oil, or water. The organic solvent may be decane and/or dodecane.

The microbial agent may be in various dosage forms, such as a liquid, an emulsion, a suspension, a powder, granules, a wettable powder, or a water-dispersible granule. According to needs, a binder, a stabilizer, etc. may also be added to the microbial agent, without specific limitation.

### Method for Enriching and Separating Lithium from Lithium-Containing Aqueous Medium

Strain HB04 of the present disclosure can grow and reproduce under conditions of 10-50 °C, pH 4.0-11.0, and salinity 0-7%, and can enrich lithium to a certain extent. Therefore, it can adapt to various lithium-containing aqueous environments, such as high-temperature or low-temperature, acidic or alkaline, and low-salinity or high-salinity lithium-containing aqueous environments. In some embodiments, the lithium-containing aqueous environment is a lithium-containing aqueous medium, such as a brine environment, which may further be oilfield produced water, and in particular heavy-oil produced water. In other embodiments, the lithium-containing aqueous medium is a lithium-containing culture medium or culture broth.

Accordingly, the present disclosure further provides use of strain HB04 or the microbial agent described above as a lithium enrichment agent, and provides use thereof for lithium enrichment and separation in a lithium-containing environment. The lithium-containing environment may be a low-grade brine environment, preferably oilfield produced water, and more preferably heavy-oil produced water.

The present disclosure provides a method for enriching and separating lithium from a lithium-containing aqueous medium. The method comprises: applying strain HB04 or the microbial agent of the present disclosure to the lithium-containing aqueous medium, such that it is contact-cultured with the lithium-containing aqueous medium under conditions suitable for growth or metabolism of strain HB04, so that lithium is adsorbed onto cells of HB04 and/or accumulated within cells of HB04 and/or metabolic products thereof; and after contact cultivation, performing solid-liquid separation.

In some embodiments, the lithium-containing aqueous medium is a brine, such as oilfield produced water, preferably heavy-oil produced water. The contact cultivation may be carried out within the ranges of 10-50 °C, pH 4.0-11.0, and salinity 0-7%; within the ranges of 10-40 °C, pH 5.0-8.0, and salinity 0-5%, the lithium accumulation amount and enrichment rate show almost no significant attenuation.

In some embodiments, when the lithium-containing aqueous medium is heavy-oil produced water, additional nutrients, such as sodium acetate, may be added during the contact cultivation. Further, the carbon-to-nitrogen ratio (C/N) of the system may be about 25 (e.g., calculated as sodium acetate).

In some embodiments, for ease of characterization and calculation, the carbon-to-nitrogen ratio (C/N) may be expressed as the mass ratio of chemical oxygen demand to nitrogen content of the system (e.g., COD/N).

In some embodiments, in addition to a carbon source (e.g., sodium acetate), the additional nutrients may further comprise a nitrogen source (e.g., ammonium chloride) and/or a phosphorus source (e.g., potassium dihydrogen phosphate).

For example, in some embodiments, about 1680 mg/L sodium acetate, about 200 mg/L ammonium chloride, and about 30 mg/L potassium dihydrogen phosphate may be added, so that COD/N is about 25.

### IV. EXAMPLES

The following examples are only used to illustrate the technical solutions of the present disclosure, and do not constitute a limitation of the present disclosure. Those of ordinary skill in the art may make various modifications or equivalent substitutions thereto without departing from the spirit and scope of the present disclosure.

The formulations of the media used in the following examples are as follows:

LB liquid medium: 10 g/L tryptone, 5 g/L yeast extract, 30 g/L sodium chloride.

LB solid medium: 10 g/L tryptone, 5 g/L yeast extract, 30 g/L sodium chloride, 18 g/L agar.

The above media correspond to a salinity of 3% (NaCl, w/v). As used herein, “salinity %” in the specification refers to salt content calculated as the mass/volume percentage (w/v) of NaCl.

Unless otherwise specified, in order to evaluate the lithium enrichment performance of strain HB04, the culture systems used in the following examples involving determination of “Li⁺ enrichment rate” and/or “Li⁺ cellular accumulation amount” are lithium-containing systems, that is, a soluble lithium salt is added to the corresponding medium to provide detectable Li⁺. For example, a lithium-containing LB medium may be prepared by adding a lithium source (e.g., lithium chloride) to an LB basic medium as described in Example 2.

### Example 1: Isolation, Deposit, Activation and Identification of Strain HB04

### (1) Isolation of Strain HB04

Antarctic sea ice was collected and a plurality of strains were isolated. By additionally adding 10 mg/L lithium chloride to the LB liquid medium, 14 strains that grew well and possessed lithium enrichment capability were preliminarily screened; further, strain HB04 with the strongest Li⁺ cellular accumulation capability and the highest enrichment rate was selected (shake-flask experimental conditions: 10 °C, 200 r/min).

### (2) Activation and Storage of Strain HB04

100 μL of stored HB04 culture was added to 50 mL LB liquid medium. After activation for 24 h, a culture with OD₆₀₀=0.5 (measured after 10-fold dilution of the culture) was obtained.

A glycerol cryopreservation method was used: 0.5 mL of the activated culture was transferred into a cryotube containing 1 mL of 30% sterilized glycerol, and stored frozen at −80 °C.

### (3) Identification of Strain HB04

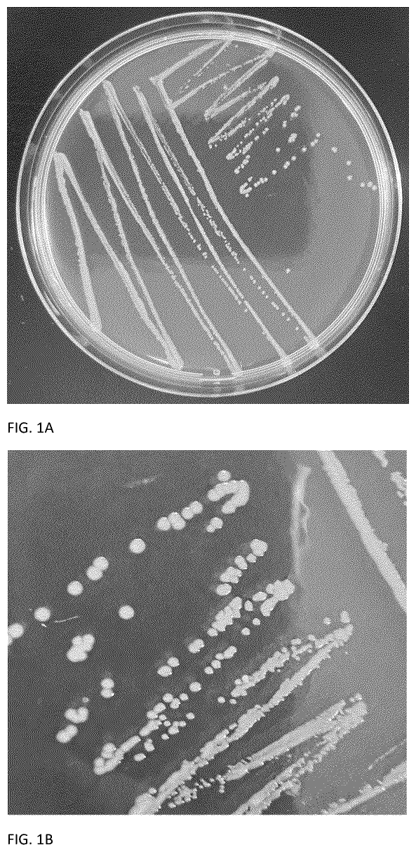

Colonies formed by strain HB04 are circular, moist, smooth, and yellow, and grow neatly along streak lines. A photograph of colonies of strain HB04 cultured on a plate is shown in FIG. 1A and FIG. 1B.

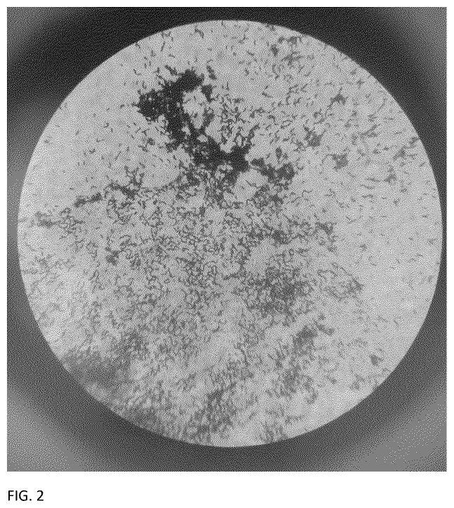

The physiological and biochemical characteristics of strain HB04 are: aerobic; Gram staining is positive; the cells are rod-shaped; some are arranged at certain angles, and two cells may form a V-shape. An optical microscope image after staining is shown in FIG. 2.

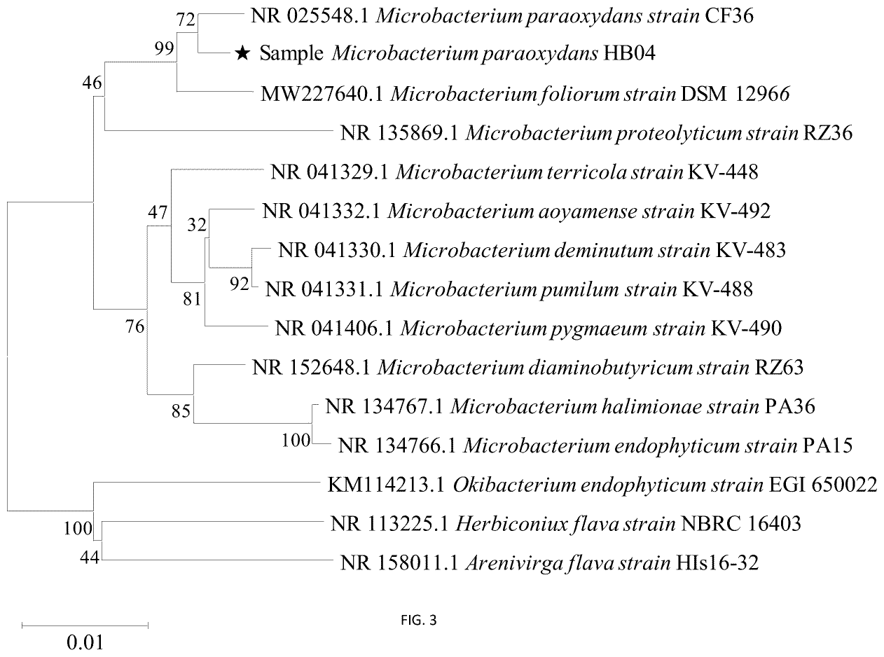

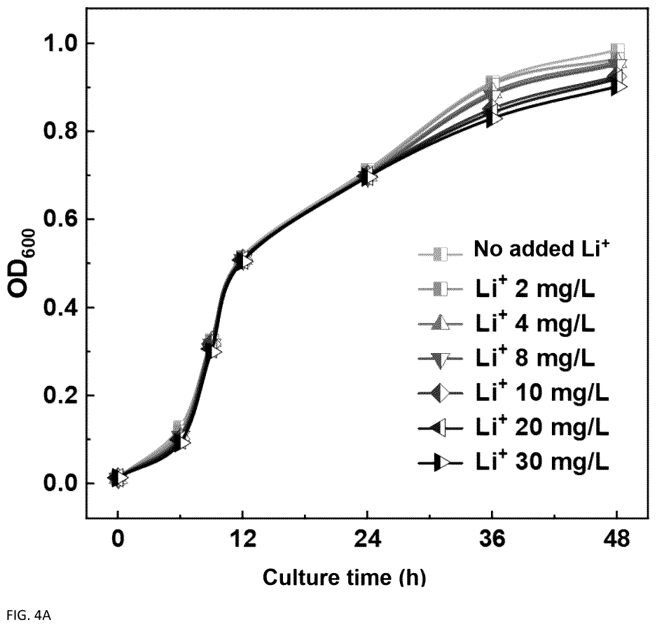

Activated strain HB04 was inoculated into LB liquid medium and cultured with shaking at 25 °C and 200 rpm for 24 h. 1 mL of fresh culture was centrifuged at 4 °C and 8000 rpm for 5 min, and the cells were collected in a 2 mL centrifuge tube. DNA was extracted using a DNA extraction kit. After electrophoresis testing, PCR amplification was performed using universal primers 7F/1540R for the 16S rDNA gene. After electrophoresis testing of the PCR product, the 16S rDNA gene sequence was determined. After sequencing, the 16S rDNA sequence of strain HB04 was uploaded to the website of the National Center for Biotechnology Information, compared with type strains on the website, and a bacterial phylogenetic tree was constructed using MEGA-X software as shown in FIG. 3. Thus, strain HB04 was determined to belong to the genus Microbacterium and was named Microbacterium paraoxydans HB04. The strain was deposited on Feb. 17, 2025 at the China Center for Type Culture Collection (CCTCC, address: No. 299 Bayi Road, Wuchang District, Wuhan City, Hubei Province, China), under accession number CCTCC NO: M 2025230.

### Example 2: Effect of Initial Li⁺ Concentration on Growth and Lithium Enrichment of Strain HB04

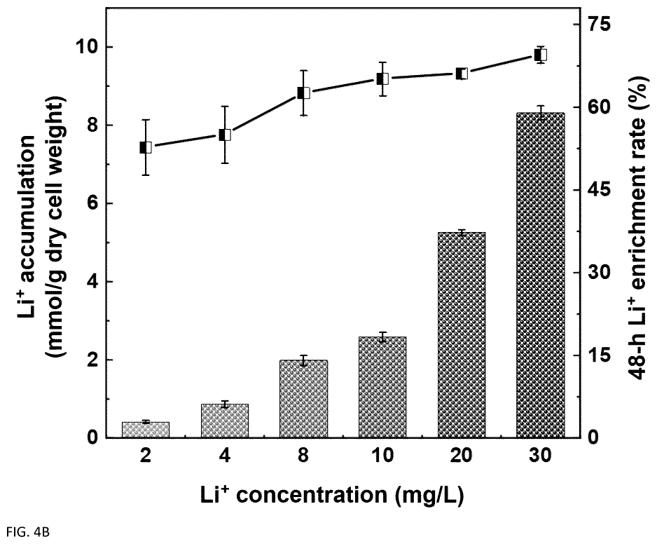

The activated culture in Example 1 was inoculated into fresh LB liquid media without Li⁺, and LB liquid media containing 2 mg/L Li⁺, 4 mg/L Li⁺, 8 mg/L Li⁺, 10 mg/L Li⁺, 20 mg/L Li⁺, and 30 mg/L Li⁺, respectively, such that the initial cell concentration was OD₆₀₀=0.013 (measured after 10-fold dilution of the culture). Three parallel samples and one blank control group without inoculation of the strain were set. The cultures were incubated in a constant-temperature incubator at 25 °C and 200 rpm. Samples were periodically taken in a clean bench to measure the cell OD values, and atomic absorption spectrometry was used to measure the Li⁺ enrichment rate at 48 h. The Li⁺ cellular accumulation amount was calculated. Results are shown in FIG. 4A and FIG. 4B.

The results indicate that within the concentration range of 0-30 mg/L, Li⁺ has almost no effect on growth of strain HB04, and the cell OD value continuously increases to above 0.9 (measured after 10-fold dilution of the culture). With an increase in Li⁺ concentration, the Li⁺ cellular accumulation amount and Li⁺ enrichment rate at 48 h increase significantly. When Li⁺ is 30 mg/L, the Li⁺ cellular accumulation amount and Li⁺ enrichment rate at 48 h reach 8.32 mmol/g (dry cell weight) and 69.57%, respectively.

### Example 3: Effect of Salinity on Growth and Lithium Enrichment of Strain HB04

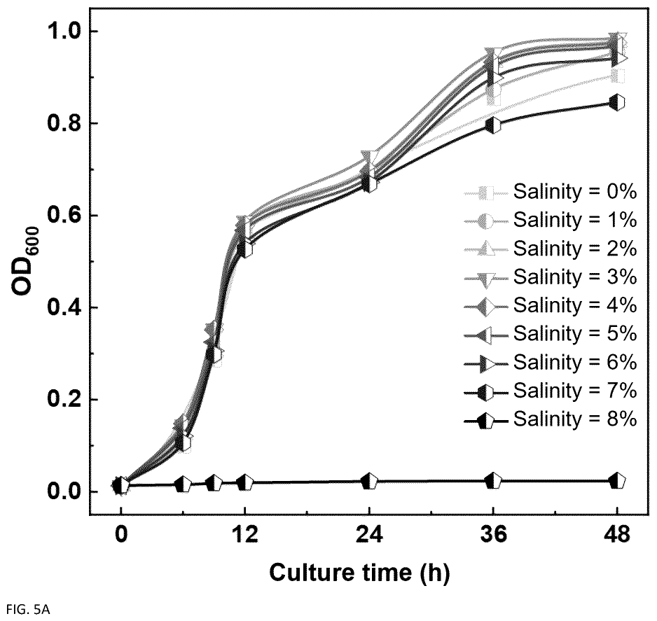

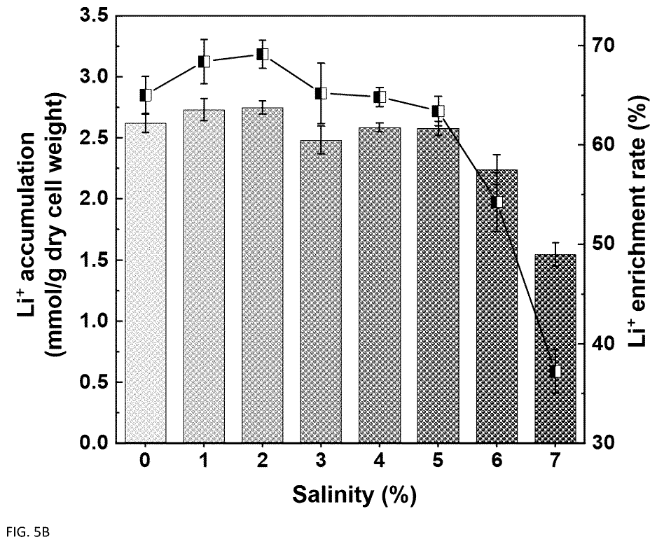

The activated culture in Example 1 was inoculated into fresh lithium-containing LB liquid media (lithium chloride was added as the lithium source so that the initial Li⁺ concentration was 10 mg/L, calculated as Li⁺), and the salinity was adjusted to 0%, 1%, 2%, 3%, 4%, 5%, 6% and 7%, respectively, such that the initial cell concentration was OD₆₀₀=0.013 (measured after 10-fold dilution of the culture). Three parallel samples and one blank control group without inoculation of the strain were set. The cultures were incubated in a constant-temperature incubator at 25 °C and 200 rpm. Samples were periodically taken in a clean bench to measure the cell OD values, and atomic absorption spectrometry was used to measure the Li⁺ enrichment rate at 48 h. The Li⁺ cellular accumulation amount was calculated. Results are shown in FIG. 5A and FIG. 5B.

The results indicate that strain HB04 can grow within the salinity range of 0-7%. The cell OD value (measured after 10-fold dilution of the culture) continuously increases to 0.8 (salinity 7%) to above 0.9 (salinity 3%), indicating good salt tolerance. Li⁺ enrichment can be achieved at salinity 0-7%. For example, at 2% salinity, the lithium enrichment rate can reach about 69%, and the Li⁺ accumulation amount of HB04 at 48 h exceeds 2.74 mmol/g (dry cell weight).

### Example 4: Effect of Reaction Temperature on Growth and Lithium Enrichment of Strain HB04

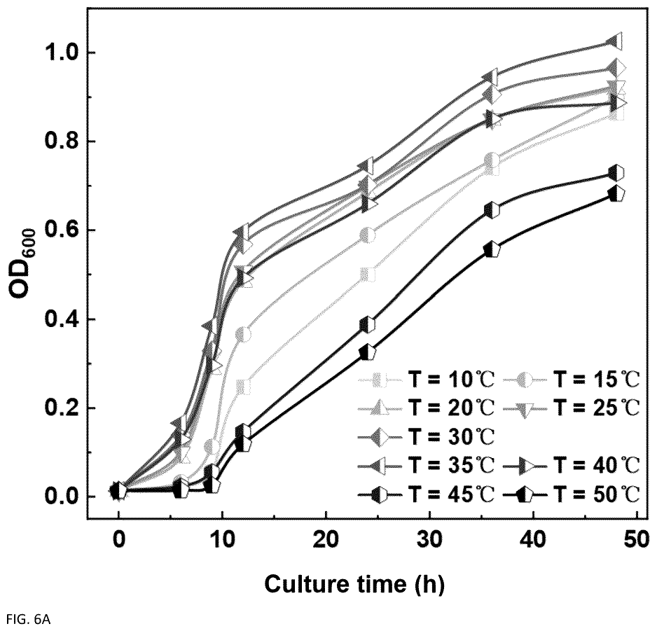

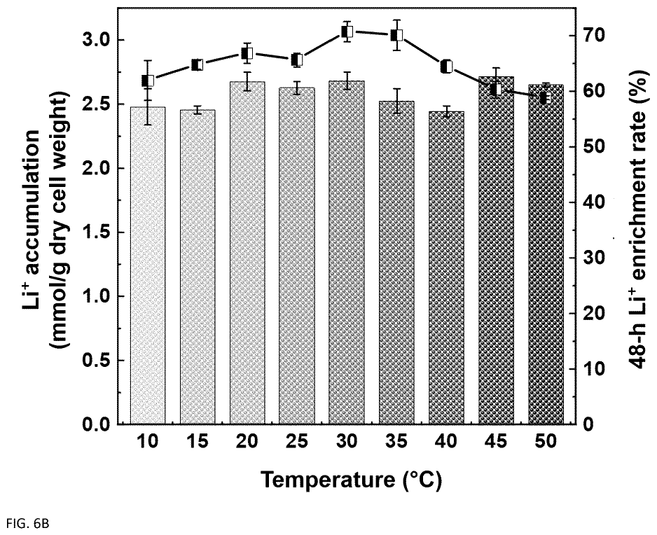

The activated culture in Example 1 was inoculated into fresh lithium-containing LB liquid medium (the initial Li⁺ concentration was 10 mg/L, calculated as Li⁺), such that the initial cell concentration was OD₆₀₀=0.013 (measured after 10-fold dilution of the culture). The cultures were placed in a constant-temperature incubator and incubated at 10 °C, 15 °C, 20 °C, 25 °C, 30 °C, 35 °C, 40 °C, 45 °C, and 50 °C, respectively, at 200 rpm. Three parallel samples and one blank control group without inoculation of the strain were set. Samples were periodically taken in a clean bench to measure the cell OD values, and atomic absorption spectrometry was used to measure the Li⁺ enrichment rate at 48 h. The Li⁺ cellular accumulation amount was calculated. Results are shown in FIG. 6A and FIG. 6B.

The results indicate that strain HB04 can grow abundantly within the temperature range of 10 °C-50 °C. The cell OD value (measured after 10-fold dilution of the culture) continuously increases to 0.6 (50 °C) to above 1.0 (35 °C), indicating good tolerance to high and low temperatures. The Li⁺ accumulation amount at 48 h remains above 2.48 mmol/g (dry cell weight), and the Li⁺ enrichment rate can reach up to 70.06%.

### Example 5: Effect of Initial pH on Growth and Lithium Enrichment of Strain HB04

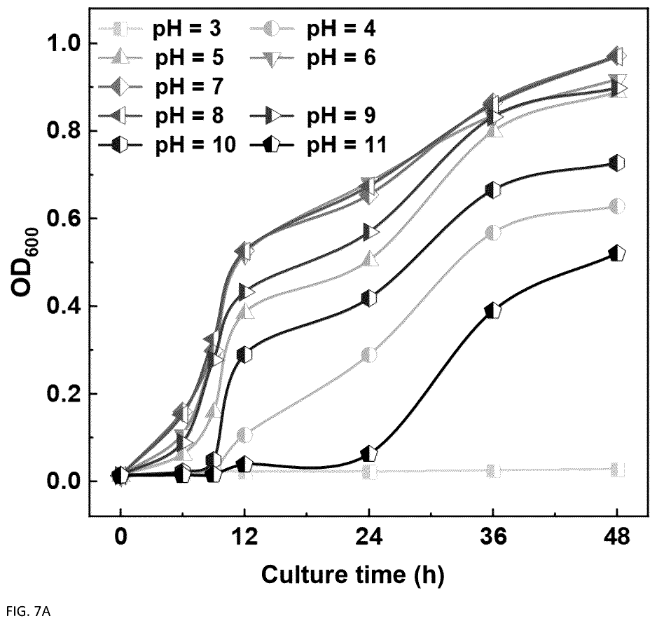

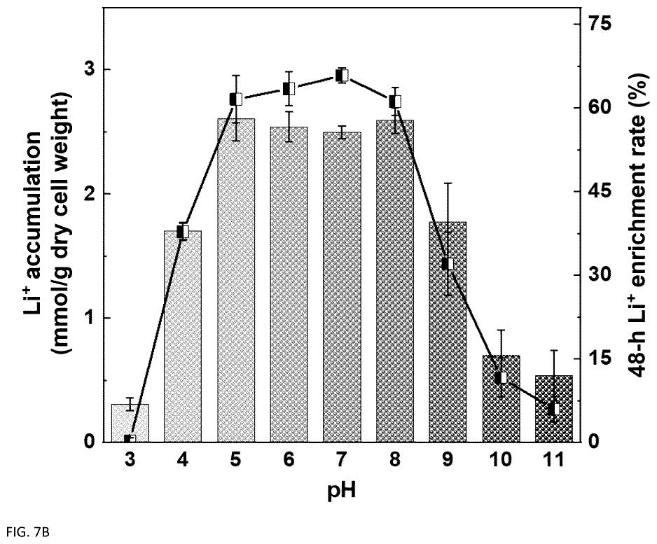

1 mol/L hydrochloric acid and 1 mol/L sodium hydroxide were used to adjust the pH of lithium-containing LB liquid medium to 3, 4, 5, 6, 7, 8, 9, 10 and 11 (the initial Li⁺ concentration was 10 mg/L, calculated as Li⁺). The activated culture in Example 1 was inoculated such that the initial cell concentration was OD₆₀₀=0.013 (measured after 10-fold dilution of the culture). The cultures were incubated in a constant-temperature incubator at 25 °C and 200 rpm. Samples were periodically taken in a clean bench to measure the cell OD values, and atomic absorption spectrometry was used to measure the Li⁺ enrichment rate at 48 h. The Li⁺ cellular accumulation amount was calculated. Results are shown in FIG. 7A and FIG. 7B.

The results indicate that strain HB04 can grow abundantly within the pH range of 4-11, indicating a wide adaptation range and good alkali tolerance, and that lithium enrichment capability shows no significant attenuation within the pH range of 5-8. The Li⁺ accumulation amount at 48 h remains above 2.50 mmol/g (dry cell weight), and the Li⁺ enrichment rate can reach up to 65.84%.

### Example 6: Growth Curve and Lithium Enrichment Efficiency of Strain HB04 in Actual Heavy-Oil Produced Water

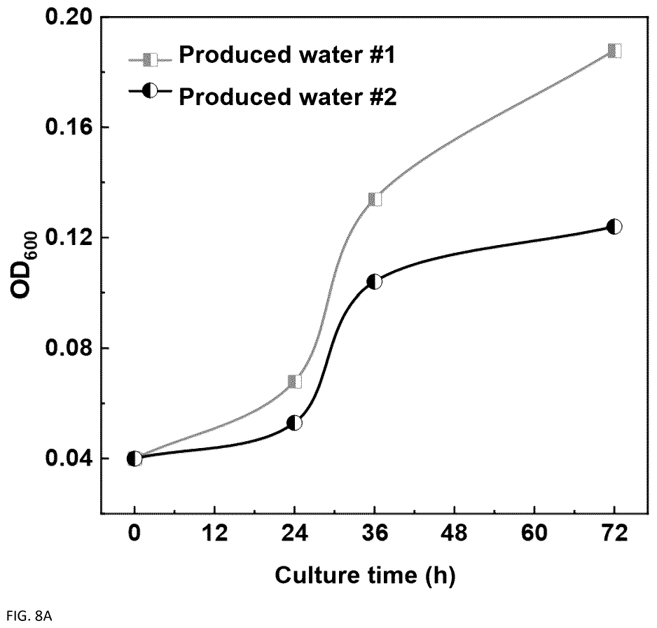

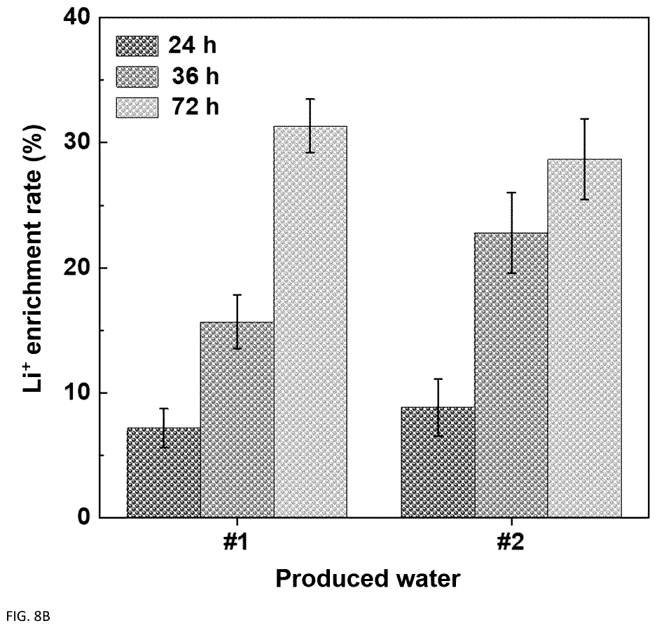

The activated culture in Example 1 was centrifuged at 8000 rpm and 4 °C for 10 min to collect the cells, which were repeatedly washed three times with 2.5% physiological saline, and inoculated at a ratio of 10% into sterilized heavy-oil produced water sample 1 and sample 2 (the lithium ion concentrations were 3.03 mg/L and 6.93 mg/L, respectively; the samples were derived from different wellheads), such that the initial cell concentration was OD₆₀₀=0.04 (measured after 10-fold dilution of the culture). Additional nutrients were added (calculated as sodium acetate, so that C/N was 25). The cultures were incubated in a constant-temperature incubator at 25 °C and 200 rpm. Samples were periodically taken in a clean bench to measure the cell OD values, and atomic absorption spectrometry was used to measure the Li⁺ enrichment rate. The Li⁺ cellular accumulation amount was calculated. Results are shown in FIG. 8A and FIG. 8B.

The results indicate that strain HB04 is capable of enriching lithium in actual heavy-oil produced water. In produced water sample 1, the Li⁺ accumulation amount at 72 h remains above 0.57 mmol/g (dry cell weight), and the Li⁺ enrichment rate is about 31.34%. In produced water sample 2, the Li⁺ accumulation amount at 72 h remains above 1.59 mmol/g (dry cell weight), and the Li⁺ enrichment rate is about 28.66%.

It should be noted that the above examples are only used to illustrate the technical solutions of the present disclosure, rather than to limit the present disclosure. Although the present disclosure has been described in detail with reference to the given examples, those of ordinary skill in the art may modify or equivalently replace the technical solutions of the present disclosure as needed without departing from the spirit and scope of the technical solutions of the present disclosure.

1. A method for enriching and separating lithium from a lithium-containing aqueous medium, comprising:
(a) contact-culturing the lithium-containing aqueous medium with a microbial agent such that lithium is adsorbed on the cells, accumulated in the cells, or both; and
(b) after the contact-culturing, separating the cells from a liquid phase to obtain lithium-containing cells;
wherein the microbial agent comprises viable cells of Microbacterium paraoxydans HB04; and
the Microbacterium paraoxydans HB04 was deposited on Feb. 17, 2025 at the China Center for Type Culture Collection (CCTCC) under accession number CCTCC NO: M 2025230.

2. The method of claim 1, wherein the lithium-containing aqueous medium is a brine.

3. The method of claim 2, wherein the brine is oilfield produced water.

4. The method of claim 3, wherein the oilfield produced water is heavy-oil produced water.

5. The method of claim 1, wherein the contact-culturing is carried out at a temperature of 10-50 °C, a pH of 4.0-11.0, and a salinity of 0-7%.

6. The method of claim 1, wherein a lithium ion concentration in the lithium-containing aqueous medium is greater than 0 mg/L and not more than 30 mg/L.

7. The method of claim 1, wherein a contact-culturing time is 24-72 h.

8. The method of claim 1, wherein, in addition to the viable cells, the microbial agent further comprises a fermentate and/or metabolic products of the Microbacterium HB04.

9. The method of claim 8, wherein the microbial agent comprises at least one selected from a fermentation broth, a culture supernatant, a cell lysate, and a cell-free extract of the Microbacterium.

10. The method of claim 8, wherein the metabolic products comprise extracellular polymers.

11. The method of claim 9, wherein the culture supernatant is a supernatant comprising extracellular metabolic products.

12. The method of claim 9, wherein the cell-free extract is a supernatant collected after centrifugation of the cell lysate.

13. The method of claim 1, wherein additional nutrients are added during the contact-culturing, and the additional nutrients comprise sodium acetate.
